(19) United States
(12) Patent Application Publication
(10) Pub. No.: US 2023/0226341 A1
(43) Pub. Date: Jul. 20, 2023

(54) PERCUTANEOUS CIRCULATORY SUPPORT DEVICE INCLUDING GUIDEWIRE DISTAL TIP PORTION

(71) Applicant: Boston Scientific Scimed Inc., Maple Grove, MN (US)

(72) Inventors: Brice Lee Shireman, Maple Grove, MN (US); Darrin Dale Beekman, Osseo, MN (US)

(21) Appl. No.: 18/099,397

(22) Filed: Jan. 20, 2023

Related U.S. Application Data
(60) Provisional application No. 63/301,114, filed on Jan. 20, 2022.

(51) Int. Cl. A61M 60/13; A61M 60/216; A61M 60/867

(52) U.S. Cl. CPC A61M 60/13; A61M 60/216; A61M 60/867

(57) ABSTRACT

A percutaneous circulatory support device includes a housing and an impeller disposed within the housing. The impeller is rotatable relative to the housing to cause blood to flow through the housing. A cannula is coupled to the housing, and a distal tip portion is coupled to the cannula opposite the housing. The distal tip portion includes an inner shaping core configured to maintain a predetermined shape of the distal tip portion and an outer layer disposed outwardly from the inner shaping core or the distal tip portion includes an atraumatic, sphere-shaped distal end.

### CROSS REFERENCE TO RELATED APPLICATION

This application claims priority to Provisional Application No. 63/301,114, filed Jan. 20, 2022, which is herein incorporated by reference in its entirety.

### TECHNICAL FIELD

The present disclosure relates to percutaneous circulatory support devices. More specifically, the present disclosure relates to percutaneous circulatory support devices including flexible distal tips.

### BACKGROUND

Percutaneous circulatory support devices such as blood pumps can provide transient support for up to approximately several weeks in patients with compromised heart function or cardiac output. Several issues may complicate delivery and operation of blood pumps within the heart, including difficulty with guidewire advancement, trauma to cardiac tissue, and oscillation and/or migration of the blood pump resulting in decreased performance of the blood pump.

### SUMMARY

In an Example 1, a percutaneous circulatory support device comprises: a housing; an impeller disposed within the housing and being rotatable relative to the housing to cause blood to flow through the housing; a cannula coupled to the housing; a distal tip portion coupled to the cannula opposite the housing, the distal tip portion comprising: an inner shaping core configured to maintain a predetermined shape of the distal tip portion; and an outer layer disposed outwardly from the inner shaping core.

In an Example 2, the percutaneous circulatory support device of Example 1, wherein the distal tip portion comprises a proximal section having a first stiffness and a distal section having a second stiffness, the second stiffness being less than the first stiffness.

In an Example 3, the percutaneous circulatory support device of either of Examples 1 or 2, wherein the inner shaping core comprises steel.

In an Example 4, the percutaneous circulatory support device of any of Examples 1-3, wherein the inner shaping core comprises a shape memory material.

In an Example 5, the percutaneous circulatory support device of any of Examples 1-4, wherein the outer layer comprises a radiopaque material.

In an Example 6, the percutaneous circulatory support device of any of Examples 1-5, wherein the distal tip portion further comprises an atraumatic, sphere-shaped distal end.

In an Example 7, the percutaneous circulatory support device of any of Examples 1-6, wherein the device is usable without an ancillary guidewire.

In an Example 8, the percutaneous circulatory support device of any of Examples 1-6, wherein the device is usable with an ancillary guidewire.

In an Example 9, a percutaneous circulatory support device comprises: a housing; an impeller disposed within the housing and being rotatable relative to the housing to cause blood to flow through the housing; a cannula coupled to the housing; and a distal tip portion coupled to the cannula opposite the housing, the distal tip portion comprising an atraumatic, sphere-shaped distal end.

In an Example 10, the percutaneous circulatory support device of Example 9, wherein the distal tip portion comprises a proximal section having a first stiffness and a distal section having a second stiffness, the second stiffness being less than the first stiffness.

In an Example 11, the percutaneous circulatory support device of either of Examples 9 or 10, wherein the distal tip portion comprises a shape memory material.

In an Example 12, the percutaneous circulatory support device of any of Examples 9-11, wherein the distal tip portion comprises steel.

In an Example 13, the percutaneous circulatory support device of any of Examples 9-12, wherein the distal tip portion comprises a radiopaque material.

In an Example 14, the percutaneous circulatory support device of any of Examples 9-13, wherein the device is usable without an ancillary guidewire.

In an Example 15, the percutaneous circulatory support device of any of Examples 9-13, wherein the device is usable with an ancillary guidewire.

In an Example 16, a percutaneous circulatory support device comprises: a housing comprising an inlet and an outlet; an impeller disposed within the housing and being rotatable relative to the housing to cause blood to flow into the inlet, through the housing, and out of the outlet; a cannula coupled to the housing; a distal tip portion coupled to the cannula opposite the housing, the distal tip portion comprising: an inner shaping core configured to maintain a predetermined shape of the distal tip portion; and an outer layer disposed outwardly from the inner shaping core.

In an Example 17, the percutaneous circulatory support device of Example 16, wherein the distal tip portion comprises a proximal section having a first stiffness and a distal section having a second stiffness, the second stiffness being less than the first stiffness.

In an Example 18, the percutaneous circulatory support device of Example 16, wherein the inner shaping core comprises steel.

In an Example 19, the percutaneous circulatory support device of Example 18, wherein the outer layer comprises a radiopaque material.

In an Example 20, the percutaneous circulatory support device of Example 19, wherein the distal tip portion further comprises an atraumatic, sphere-shaped distal end.

In an Example 21, the percutaneous circulatory support device of Example 18, wherein the distal tip portion further comprises an atraumatic, sphere-shaped distal end.

In an Example 22, the percutaneous circulatory support device of Example 16, wherein the distal tip portion further comprises an atraumatic, sphere-shaped distal end.

In an Example 23, the percutaneous circulatory support device of Example 22, wherein outer layer comprises a radiopaque material.

In an Example 24, the percutaneous circulatory support device of Example 16, wherein outer layer comprises a radiopaque material.

In an Example 25, a percutaneous circulatory support device comprises: a housing comprising an inlet and an outlet; an impeller disposed within the housing and being rotatable relative to the housing to cause blood to flow into the inlet, through the housing, and out of the outlet; a cannula coupled to the housing; and a distal tip portion coupled to the cannula opposite the housing, the distal tip portion comprising an atraumatic, sphere-shaped distal end.

In an Example 26, the percutaneous circulatory support device of Example 25, wherein the distal tip portion comprises a proximal section having a first stiffness and a distal section having a second stiffness, the second stiffness being less than the first stiffness.

In an Example 27, the percutaneous circulatory support device of Example 25, wherein the distal tip portion comprises a shape memory material.

In an Example 28, the percutaneous circulatory support device of Example 25, wherein the distal tip portion comprises steel.

In an Example 29, the percutaneous circulatory support device of Example 25, wherein the distal tip portion comprises a radiopaque material.

In an Example 30, a method for positioning a blood pump within a subject, the blood pump comprises a cannula and a distal tip portion coupled to the cannula, the distal tip portion comprising an inner shaping core configured to maintain a predetermined shape of the distal tip portion and an outer layer disposed outwardly from the inner shaping core, and the method comprises: advancing the blood pump through the vasculature of the subject; and crossing the aortic valve of the subject with the blood pump such that the distal tip portion is positioned in the left ventricle of the subject.

In an Example 31, The method of Example 30, wherein advancing the blood pump through the vasculature of the subject comprises advancing the blood pump without using an ancillary guidewire.

In an Example 32, The method of Example 30, wherein advancing the blood pump through the vasculature of the subject comprises advancing the distal tip portion along an ancillary guidewire.

In an Example 33, The method of Example 30, wherein crossing the aortic valve comprises configuring the distal tip portion in a prolapsed configuration.

In an Example 34, The method of Example 30, wherein the distal tip portion comprises a proximal section having a first stiffness and a distal section having a second stiffness, the second stiffness being less than the first stiffness.

In an Example 35, The method of Example 30, wherein the distal tip portion further comprises an atraumatic, sphere-shaped distal end.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a partial side view of an illustrative percutaneous circulatory support device (also referred to herein, interchangeably, as a “blood pump”) positioned in the aorta and the heart of a patient, in accordance with embodiments of the subject matter disclosed herein.

FIG. 2 is another partial side view of the percutaneous circulatory support device of FIG. 1.

FIG. 3 is a partial side sectional view of the percutaneous circulatory support device of FIG. 1.

FIG. 4 is a partial side view of another illustrative percutaneous circulatory support device, in accordance with embodiments of the subject matter disclosed herein.

FIG. 5 is a partial side sectional view of yet another percutaneous circulatory support device, in accordance with embodiments of the subject matter disclosed herein.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

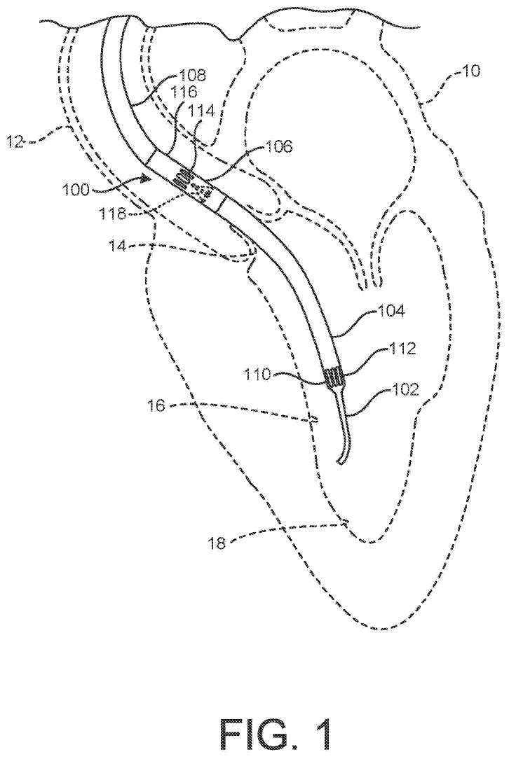

FIG. 1 depicts a portion of an illustrative percutaneous mechanical circulatory support device 100 (also referred to herein, interchangeably, as a “blood pump”), and its relative position in a human heart 10, in accordance with embodiments of the subject matter disclosed herein. The blood pump 100 may be delivered percutaneously by passing through the aorta 12 and positioned with the heart 10 with respect to the aortic valve 14 and the left ventricle 16, as shown in FIG. 1. In some embodiments and as described in further detail below, the blood pump 100 may provide enhanced trackability and may be delivered without using an ancillary guidewire (not shown—that is, a guidewire separate from the blood pump 100). Alternatively, the blood pump 100 may be delivered using an ancillary guidewire.

With continued reference to FIG. 1, the blood pump 100 generally includes a flexible distal tip portion 102 (which may also be referred to as a “guidewire tip”), a cannula 104, an impeller portion 106, and a catheter 108. The cannula 104 may have a flexible construction to facilitate delivery of the blood pump 100. The cannula 104 includes one or more blood inlets 110 located on a distal portion 112 thereof, and one or more blood outlets 114 are located on a housing 116 of the impeller portion 106. The housing 116 carries an impeller 118, and the impeller 118 rotates relative to the housing 116 to cause blood to flow into the inlets 110, through the housing 116, and out of the outlets 114. During operation and as shown in FIG. 1, the blood pump 100 is positioned within the heart 10 such that the inlets 110 are positioned in the left ventricle 16 and the outlets 114 are positioned in the aorta 12. As a result, rotation of the impeller 118 relative to the housing 116 causes blood to flow from the left ventricle 16, through the cannula 104 and the impeller portion 106, and into the aorta 12. The flexible distal tip portion 102 is described in further detail below. However, during operation, the blood pump 100 may be positioned such that the distal tip portion 102 is located in close proximity of, or in contact with, the wall of the left ventricle 16, for example, in the location of the apex 18 of the left ventricle 16.

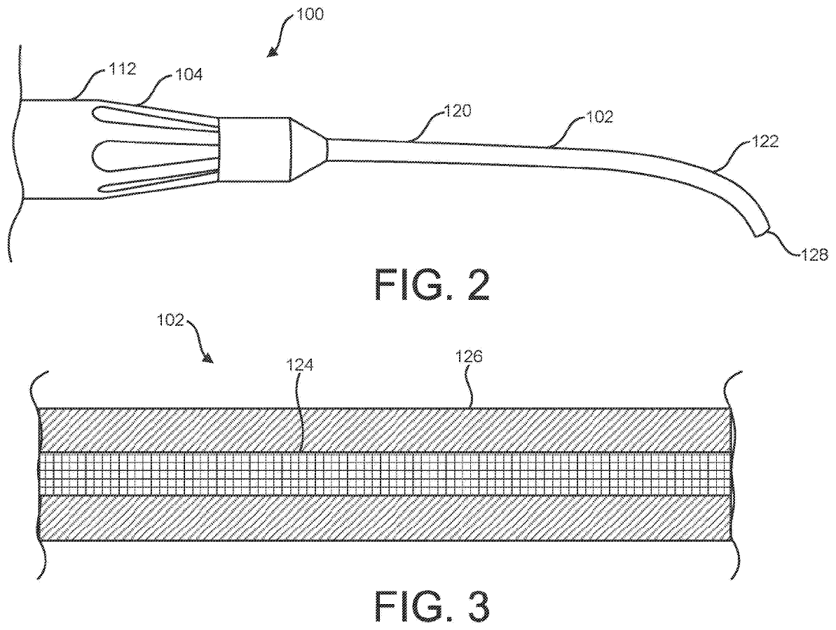

FIG. 2 depicts a side view of a portion of the blood pump 100, specifically the distal portion 112 of the cannula 104 and the distal tip portion 102. The distal tip portion 102 includes a proximal section 120 adjacent to the cannula 104 and a distal section 122 adjacent to the proximal section 120 and opposite the cannula. In some embodiments and as illustrated, the proximal section 120 has a generally straight shape and the distal section 122 has a generally curved shape. In other embodiments, the proximal section 120 and/or the distal section 122 have different shapes.

In some embodiments, the proximal section 120 of the distal tip portion 102 is constructed to have a relatively high stiffness (compared to the distal section 122) so that it may withstand forces acting on the distal tip portion 102 and the blood pump 100. Such a stiffness also provides axial strength, which facilitates positioning and supporting the cannula 104 in the left ventricle. The stiffness of proximal section 120 may be achieved by constructing the proximal section 120 of one or more materials of appropriate hardness, by the inclusion of structures, such as reinforcement structures or slots, within the proximal section 120, by combining materials and structures to achieve the appropriate stiffness, and/or by using other techniques known to those of ordinary skill in the art.

In some embodiments, the distal section 122 of the distal tip portion 102 is constructed to have a relatively low stiffness (compared to the proximal section 120). Such a stiffness facilitates atraumatic contact with tissue yet provides adequate structural strength for positioning and supporting the cannula 104 in the left ventricle while also being capable of absorbing forces acting on the distal tip portion 102. The stiffness of the distal section 122 may be achieved by constructing the distal section 122 of one or more materials of appropriate hardness, by the inclusion of structures, such as reinforcement structures or slots, within the distal section 122, by combining materials and structures to achieve the appropriate stiffness, and/or by using other techniques known to those of ordinary skill in the art. In general, the distal section 122 may be constructed of materials that have a stiffness less than the stiffness of the materials forming proximal section 120, as measured, for example, by a durometer. In some embodiments, based on the materials used for the reinforcement structures, the inclusion of the structures may aid in the visualization for the distal tip portion 102 under fluoroscopy.

As described above, the proximal section 120 of the distal tip portion 102 may have a greater stiffness than the distal section 122 of the distal tip portion 102. In some embodiments, one or more stiffness transitions may occur in discrete steps along the length of the distal tip portion 102. In some embodiments, one or more stiffness transitions may be gradual or continuous along the length of the distal tip portion 102. In other embodiments, one or more stiffness transitions may be a combination of discrete steps and continuous segments. In some embodiments, one or more stiffness transitions may be achieved by decreasing the wall thickness of the distal tip portion 102 from the proximal section 120 to the distal section 122, decreasing the stiffness of material along the length of distal tip portion 102 without using discrete segments, or by any other methods known to those of ordinary skill in the art.

FIG. 3 depicts a side sectional view of a section of the distal tip portion 102. In some embodiments and as illustrated, the distal tip portion 102 includes multiple components or layers. More specifically, the distal tip portion 102 may include an inner shaping core 124 and an outer layer 126 disposed outwardly of the inner shaping core 124. The inner shaping core 124 may be constructed of one or more materials that are configured to maintain a predetermined shape of the distal tip portion 102 (for example, shape memory materials). In some embodiments, the inner shaping core 124 may be constructed of one or more metals, such as steel, for example spring steel or stainless steel. In some embodiments, the inner shaping core 124 may be constructed of one or more materials that are configured to be shaped by a user, such as a medical practitioner. In some embodiments, the inner shaping core 124 may be a stamped ribbon. In some embodiments, the outer layer 126 may be constructed of one or more polymers (for example, polyether block amide, thermoplastic polyurethane, or the like). In some embodiments, the outer layer 126 may be constructed of one or more radiopaque materials.

The distal tip portion 102 may provide one or more of various further advantages. For example, if the cannula 104 is constructed to be flexible, blood flow and contraction of the heart may cause movement or oscillation of the blood pump 100 within the heart. The flexible distal tip portion 102 may account for and reduce or counteract the lateral contraction forces acting on the distal tip portion 102 during contraction of the left ventricle. The distal tip portion 102 may significantly reduce such movement or oscillation, for example by contacting a wall or surface of the left ventricle, thereby stabilizing the entire blood pump 100. Such stabilization of the blood pump 100 may increase efficiency, performance, and/or longevity of the blood pump 100. As another example, the distal tip portion 102 may facilitate crossing the aortic valve in a prolapsed configuration of the distal tip portion 102 (that is, with a distal end 128 (FIG. 2) of the distal tip portion 102 facing away from the aortic valve). As a further example, inclusion of the distal tip portion 102 as described above may allow for the blood pump 100 to be advanced through the vasculature of the subject, cross the aortic valve, and be delivered to the left ventricle without using an ancillary guidewire.

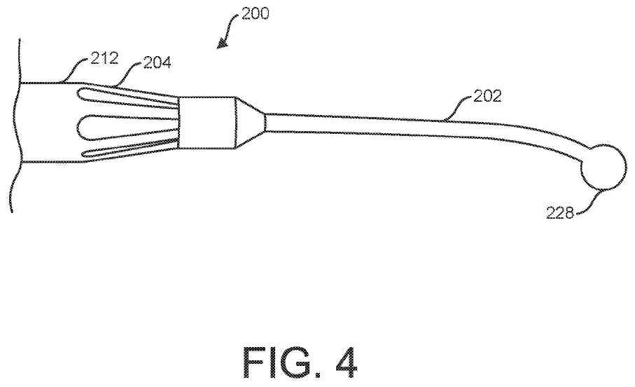

FIG. 4 depicts a side view of a portion of a blood pump 200, in accordance with embodiments of the subject matter disclosed herein. Specifically, FIG. 4 depicts a distal portion 212 of a cannula 204 and a distal tip portion 202 of the blood pump 200. The cannula 204 may be the same as or similar to the cannula 104 described above. The distal tip portion 202 may be the same as or similar to the distal tip portion 102 described above, except that the distal tip portion 202 includes an enlarged, atraumatic distal end 228. Specifically, the distal tip portion 202 includes an atraumatic, sphere-shaped distal end 228. As illustrated, the sphere-shaped distal end 228 may have a relatively large diameter compared to a diameter or width of the remainder of the distal tip portion 202.

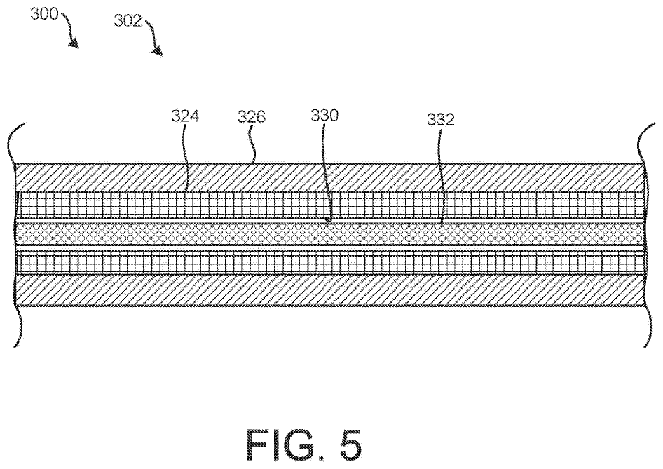

In some embodiments, a distal tip portion may include a lumen to facilitate passage of an ancillary guidewire. For example and referring now to FIG. 5, a distal tip portion 302 of a blood pump 300 is illustrated, in accordance with embodiments of the subject matter disclosed herein. The distal tip portion 302 includes an inner shaping core 324 and an outer layer 326 disposed outwardly of the inner shaping core 324. The inner shaping core 324 includes an inner lumen 330 that is capable of receiving an ancillary guidewire 332. In some embodiments, the ancillary guidewire 332 may straighten the curved shape of the distal tip portion 302. As an additional or alternative feature, a distal tip portion may include one or more active steering devices (not shown—such as push wires, pull wires, or the like) for straightening its curved shape. In either case, such features facilitate advancing and properly positioning the distal tip portion and the blood pump within a subject.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A percutaneous circulatory support device, comprising:
a housing comprising an inlet and an outlet;
an impeller disposed within the housing and being rotatable relative to the housing to cause blood to flow into the inlet, through the housing, and out of the outlet;
a cannula coupled to the housing;
a distal tip portion coupled to the cannula opposite the housing, the distal tip portion comprising:
an inner shaping core configured to maintain a predetermined shape of the distal tip portion; and
an outer layer disposed outwardly from the inner shaping core.

2. The percutaneous circulatory support device of claim 1, wherein the distal tip portion comprises a proximal section having a first stiffness and a distal section having a second stiffness, the second stiffness being less than the first stiffness.

3. The percutaneous circulatory support device of claim 1, wherein the inner shaping core comprises steel.

4. The percutaneous circulatory support device of claim 3, wherein the outer layer comprises a radiopaque material.

5. The percutaneous circulatory support device of claim 4, wherein the distal tip portion further comprises an atraumatic, sphere-shaped distal end.

6. The percutaneous circulatory support device of claim 3, wherein the distal tip portion further comprises an atraumatic, sphere-shaped distal end.

7. The percutaneous circulatory support device of claim 1, wherein the distal tip portion further comprises an atraumatic, sphere-shaped distal end.

8. The percutaneous circulatory support device of claim 2, wherein outer layer comprises a radiopaque material.

9. The percutaneous circulatory support device of claim 1, wherein outer layer comprises a radiopaque material.

10. A percutaneous circulatory support device, comprising:
a housing comprising an inlet and an outlet;
an impeller disposed within the housing and being rotatable relative to the housing to cause blood to flow into the inlet, through the housing, and out of the outlet;
a cannula coupled to the housing; and
a distal tip portion coupled to the cannula opposite the housing, the distal tip portion comprising an atraumatic, sphere-shaped distal end.

11. The percutaneous circulatory support device of claim 10, wherein the distal tip portion comprises a proximal section having a first stiffness and a distal section having a second stiffness, the second stiffness being less than the first stiffness.

12. The percutaneous circulatory support device of claim 10, wherein the distal tip portion comprises a shape memory material.

13. The percutaneous circulatory support device of claim 10, wherein the distal tip portion comprises steel.

14. The percutaneous circulatory support device of claim 10, wherein the distal tip portion comprises a radiopaque material.

15. A method for positioning a blood pump within a subject, the blood pump comprising a cannula and a distal tip portion coupled to the cannula, the distal tip portion comprising an inner shaping core configured to maintain a predetermined shape of the distal tip portion and an outer layer disposed outwardly from the inner shaping core, and the method comprising:
advancing the blood pump through the vasculature of the subject; and
crossing the aortic valve of the subject with the blood pump such that the distal tip portion is positioned in the left ventricle of the subject.

16. The method of claim 15, wherein advancing the blood pump through the vasculature of the subject comprises advancing the blood pump without using an ancillary guidewire.

17. The method of claim 15, wherein advancing the blood pump through the vasculature of the subject comprises advancing the distal tip portion along an ancillary guidewire.

18. The method of claim 15, wherein crossing the aortic valve comprises configuring the distal tip portion in a prolapsed configuration.

19. The method of claim 15, wherein the distal tip portion comprises a proximal section having a first stiffness and a distal section having a second stiffness, the second stiffness being less than the first stiffness.

20. The method of claim 15, wherein the distal tip portion further comprises an atraumatic, sphere-shaped distal end.
